(19) United States
(12) Patent Application Publication
(10) Pub. No.: US 2022/0362141 A1
(43) Pub. Date: Nov. 17, 2022

(54) A STABLE EFFERVESCENT CO-PROCESSED EXCIPIENT COMPOSITION AND A PROCESS FOR PREPARING THE SAME

(71) Applicant: ISP INVESTMENTS LLC, Wilmington, DE (US)

(72) Inventors: Desai MAHESH, Parsippany, NJ (US); Robert MCCRIMLISK, Sparta, NJ (US); Quyen Vo SCHWING, Mount Laurel, NJ (US)

(73) Assignee: ISP INVESTMENTS LLC, Wilmington, DE (US)

(21) Appl. No.: 17/769,679

(22) PCT Filed: Oct. 13, 2020
(86) PCT No.: PCT/US2020/055400

§ 371 Date: Apr. 15, 2022

Related U.S. Application Data
(60) Provisional application No. 62/916,402, filed on Oct. 17, 2019.

(51) Int. Cl. A61K 9/46; A61K 9/16; A61K 9/20

(52) U.S. Cl. CPC A61K 9/0007; A61K 9/1611; A61K 9/1623; A61K 9/2013; A61K 9/2018

(57) ABSTRACT

A dry stable effervescent co-processed excipient composition comprising: (i) about 0.1 to about 50 wt. % of one or more carbonate bases selected from the group consisting of alkali carbonate, alkali bicarbonate, alkaline earth carbonate, alkaline earth bicarbonate and mixtures thereof; (ii) about 0.1 to about 50 wt. % of a water soluble carbohydrate sugar alcohol and; (iii) about 0.001. to about 40 wt % of one or more organic acids. Also disclosed is a process for preparing the composition.

### FIELD OF THE INVENTION

The present application relates to a dry stable effervescent co-processed excipient composition comprising: (i) about 0.1 to about 50 wt. % of one or more carbonate bases: (ii) about 0.1 to about 50 wt. % of a water soluble carbohydrate sugar alcohol and; (iii) about 0.001 to about 40 wt. % of one or more organic acids. The composition is shelf stable in standard product packaging and useful in standard ready to mix beverages.

### BACKGROUND OF THE INVENTION

Effervescent dosage forms are well known and accepted as patient and consumer centric oral delivery systems for nutritional supplements and medicines. To achieve effervescence, the appropriate stoichiometric proportions of at carbonate base such as sodium or potassium bicarbonate and a suitable organic acid such as citric, malic or tartaric acid is combined in the presence of water. The acid and base react vigorously to release CO₂. In the form of bubbles or fizzing which is generally associated with an enhanced sensory experience by the consumer. Such dosage forms can be formulated into granules which are packaged into sachets and dispersed in a glass of water at the time of use or compressed into effervescent tablets which can be dissolved into a glass of water resulting in an appealing carbonated beverage.

Current effervescent products require extreme low humidity processing and packaging environments for commercial products. Expensive equipment and packaging components are required with high rates of failure due to early unwanted reaction of the formulations.

While such dosage forms are desired and convenient, the effervescent reaction can also be triggered by environmental moisture, i.e., humidity present in the air. This makes manufacturing effervescent dosage forms highly specialized and costly as plants need to be equipped with suitable Heating Ventilation and Air Conditioning (HVAC) equipment that can maintain relative humidity below 30%, and ideally below 25% year-round. Additionally, the finished dosage forms need to be packaged in suitable moisture resistant packaging such as multilayered resistant laminated foil sachets or aluminum tubes and desiccants to hold the tablets. A need therefore exists for a ready to use stabilized co-processed excipient composition that has effervescent properties and can be used to make dosage forms that do not require special packaging and, furthermore, can be handled in manufacturing plants with conventional HVAC systems which maintain relative humidity (RH) between 30-55% depending on season and weather conditions rather than specialized humidity controlling HVAC systems.

U.S. Pat. No. 5,709,886A describes a process for microencapsulating a finely divided admixture of sodium bicarbonate and citric acid to produce a taste masked effervescent material comprising individual microcapsules each containing an effervescent mixture of sodium bicarbonate and citric acid encapsulated with ethylcellulose. The process comprises forming a granulate admixture of sodium bicarbonate and citric acid, and charging the admixture of sodium bicarbonate and citric acid to a coacervating medium that includes cyclohexane as a solvent, ethylcellulose as an encapsulating polymer, and a phase inducing polymer, and separating the microcapsules.

WO 1995023594A1 describes a granular product or tablet containing an effervescent system and an active pharmaceutical substance and a method for its preparation.

We have unexpectedly found that sodium bicarbonate and potassium bicarbonate can be granulated with mannitol, which is a water soluble carbohydrate sugar alcohol and blended with citric acid co-processed with water-soluble mannitol to yield a co-processed excipient composition which is stable in atmospheric ambient conditions under open dish conditions and in simple polyethylene bags at 40° C. and 75% relative humidity for extended periods of time.

The present application discloses a dry stable effervescent granulation that is stable even after two months and is free of any caking or clumping which is a problematic indicator in effervescent blends.

### SUMMARY OF THE INVENTION

An objective of the present application is to provide a dry stable effervescent co-processed excipient composition comprising: (i) about 0.1 to about 50 wt. % of one or more carbonate bases selected from the group consisting of alkali carbonate, alkali bicarbonate, alkaline earth carbonate, alkaline earth bicarbonate and mixtures thereof; (ii) about 0.1 to about 50 wt. % of one or more water soluble carbohydrate sugar alcohols selected from the group consisting of mannitol, maltitol, lactitol, xylitol, erythritol and mixtures thereof and; (iii) about 0.001 to about 40 wt. % of one or more organic acids.

According to one aspect of the present application, there is provided an effervescent co-processed excipient composition used for (i) enhancing stability of the composition (ii) developing free flowing; and highly compactible compositions and, (iii) developing effervescent formulations that are shelf stable in a standard product packaging. The compositions and formulations can be used in standard ready to mix beverages.

Another aspect of the present application discloses a process for preparing a co-processed finely divided admixture comprising individual particle of an effervescent carbonate base or mixture of carbonate bases encapsulated with mannitol, wherein the process comprises admixing co-processed base or bases with mannitol in solution at 20 to 30% solids and spraying onto the surface of the base or base-mix in an amount sufficient to achieve about an 8:2 weight ratio gain to provide a barrier to prevent premature reaction, drying the resultant encapsulate and dry blending with citric acid co-processed with mannitol.

Yet another aspect of the present application provides an oral solid dosage form in the Rum of tablets, capsules, pellets, granules or a sachet comprising: (i) about 0.1 to about 50 wt. % of one or more carbonate bases selected from the group consisting of sodium bicarbonate (NaHCO₃), potassium bicarbonate (KHCO₃), sodium carbonate (Na₂CO₃), potassium carbonate (K₂CO₃) and combinations thereof; (ii) about 0.1 to about 50 wt. % of mannitol; (iii) about 0.001 to about 40 wt. % of one or more organic acids; (iv) about 1 to about 3 wt. % of one or more sweeteners selected from the group consisting of stevia, aspartame, sucralose and saccharin; and (v) about 0.1 to about 1 wt. % of one or more food grade oils or flavors selected from the group consisting of avocado, coconut, palm, olive, corn, sunflower, almond, canola, berry, cherry, passion fruit, orange, blue ice, tropical fruit, raspberry, lemon and mixtures thereof.

### BRIEF DESCRIPTION OF THE FIGURES

Further embodiments of the present: application can be understood with reference to the appended figures.

FIG. 1 shows very uniform particle size distribution of the Stable effervescent co-processed excipient measured by using Malvern Mastersizer 3000 at the laser power of 71.35% with the beam length of 10 mm.

FIG. 2 represents powder flow measured by the flow function parameter using a Brookfield powder flow tester. A larger flow function indicates better flow.

FIG. 3 shows the moisture sorption isotherm for the stable effervescent co-processed excipient obtained using TGA Q5000 Instrument with the method log as follows: humidity increases from 0% to 70% at 25° C.

FIG. 4 shows that stable effervescent co-processed excipient is stable at ambient conditions stored in an open dish for 5 days and remains stable packaged in a simple Polyethylene bags when stored at 25° C., 60% RH and 40° C., 75% RH for 5 days.

FIG. 5 represents tablet hardness of each electrolyte effervescent tablet formulation tested using Natoli automated tablet hardness tester.

FIG. 6 shows cherry extract effervescent tablets have similar tablet hardness of 18 kp and disintegration time of 120 seconds, confirming effervescent tablet formulations with stable effervescent co-processed excipient are very stable and short disintegration time.

FIG. 7 shows morphology of stable effervescent granulation are well coated particle with moisture protective barrier using Scanning Electron Microscopy (SEM).

FIG. 8 shows pH of stability samples have minimal change in pH, indicating stable effervescent granulation maintained its chemical properties using the USP 791 method.

FIG. 9 shows water activity of packaged stability samples, the 40° C./75% RH samples had the highest water activity, indicating the stable effervescent granulation is most stable at room temperature (warehouse) or 25° C./60% RH conditions using the USP 922 water activity method.

FIG. 10 shows tablet characterization of the tablets with the stable effervescent granulation maintain hardness and disintegration time.

### DETAILED DESCRIPTION OF THE INVENTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in many ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed herein can be made and executed without undue experimentation in light of the present disclosure. While the articles and methods of the present disclosure have been described in terms of preferred embodiments, it will be apparent to those of ordinary skill in the art that variations may be applied to the articles and/or methods and in the steps or in the sequence of steps of the method(s) described herein without departing from the concept, spirit and scope of the present disclosure. All such similar substitutes and modifications apparent to those skilled in the art are deemed to be within the spirit, scope and concept of the present disclosure.

As utilized in accordance with the present disclosure, the following terms, unless otherwise indicated, shall be understood to have the following meanings.

The use of the word “a” or “an” when used in conjunction with the term “comprising” may mean “one,” but it is also consistent with the meaning of “one or more,” “at least one,” and “one or more than one.” The use of the term “or” is used to mean “and/or” unless explicitly indicated to refer to alternatives only if the alternatives are mutually exclusive, although the disclosure supports a definition that refers to only alternatives and “and/or.” Throughout this application, the term “about” is used to indicate that a value includes the inherent variation of error for the quantifying device, the method(s) being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 1, 2, 3, 4, 5, 10, 15, 20, 30, 40, 50, 100, etc. The term “at least one” or “at least two” may extend up to 100 or 1000 or more depending on the term to which it is attached. In addition, the quantities of 100/1000 are not to be considered limiting as lower or higher limits may also produce satisfactory results. In addition, the use of the term “at least one of X, Y, and Z” will be understood to include X alone, Y alone, and Z alone, as well as any combination of X, Y, and Z, The use of ordinal number terminology (i.e., “first”, “second”, “third”, “fourth”, etc.) is solely for the, purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The terms “or combinations thereof” and “and/or combinations thereof” as used herein refer to all permutations and combinations of the listed items preceding the term. For example, “A, B, C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more items or terms, such as BB, AAA, AAB, BBC, AAABCCCC, CBBAAA, CABABB, and so forth. The skilled artisan will understand that typically there is no limit on the number of items or terms in any combination, unless otherwise apparent from the context.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

According to one embodiment of the present application, there is provided a dry stable effervescent co-processed excipient composition comprising: (i) about 0.1 to about 50 wt. % of one or more carbonate bases selected from the group consisting of alkali carbonate, alkali bicarbonate, alkaline earth carbonate, alkaline earth bicarbonate and mixtures thereof; (ii) about 0.1 to about 50 wt. % of one or more sugar alcohol selected from the group consisting of mannitol, maltitol, lactitol, xylitol, erythritol and mixtures thereof, and (iii) about 0.001 to about 40 wt. % of one or more organic acids.

In one embodiment of the present application, the carbonate base is selected from the group consisting of alkali carbonate, alkali bicarbonate, alkaline earth carbonate, alkaline earth bicarbonate and mixtures thereof.

In another embodiment of the present application, the one or more carbonate bases can be selected from the group consisting of sodium bicarbonate (NaHCO₃), potassium bicarbonate (KHCO₃), sodium carbonate (Na₂CO₃), potassium carbonate (K₂CO₃) and combinations thereof.

In some embodiments, the carbonate base is present in suitable amounts ranging from about 0.1 wt. % to about 1 wt. %, or from about 1 wt. % to about 5 wt. %, or from about 5 wt. % to about 10 wt. % or from about 10 wt. % to about 20 wt. %, or from about 20 wt. % to about 30 wt. %, or from about 30 wt. % to about 40 wt. %, or from about 40 wt. % to about 50 wt. % based on the total weight of the composition of the present application.

In yet another embodiment of the present application, the water-soluble carbohydrate sugar alcohol is selected from the group consisting of mannitol, maltitol, lactitol, xylitol, erythritol and combinations thereof. In a non-limiting embodiment, the water-soluble carbohydrate sugar alcohol is mannitol.

In some embodiments, the water-soluble carbohydrate sugar alcohol is mannitol present in suitable amounts ranging from about 0.1 wt. % to about 1 wt. %, or from about 1 wt. % to about 5 wt. %, or from about 5 wt. % to about 10 wt. % or from about 10 wt. % to about 20 wt. % or from about 20 wt. %, to about 30 wt. %, or from about 30 wt. % to about 40 wt. %, or from about 40 wt. % to about 50 wt % based on the total weight of the composition of the present application.

In one embodiment of the present application, the organic acid is edible and selected from the group consisting of citric acid, malic acid and tartaric acid.

In some embodiments, the organic acids is/are present in suitable amounts ranging from about 0.001 wt. % to about 0.01 wt. % from about 0.01 wt. %, to about 0.1 wt. %, or from about: 0.1 wt. %, to about 1 wt %, from about 0.1 wt. % to about 1 wt. %, or from about 1 wt. % to about 5 wt. %, or from about 5 wt. % to about 10 wt. % or from about 10 wt. % to about 20 wt. %, or from about 20 wt. % to about 30 wt. %, or from about 30 wt. % to about 40 wt. % based on the total weight of the composition of the present application.

In another embodiment, the composition of the present application composes one or more sweetening agents selected from the group including, but not limited to, stevia, sucrose, glucose, saccharin, levulose, lactose, mannitol, sorbitol, fructose, maltose, xylitol, saccharin salts, thaumatin, aspartame, dihydrochalcones, acesulfame, sucralose, cyclamate salts, sodium cyclamate, sodium saccharin, and mixtures thereof In a non-limiting embodiment, the composition contains from about 1 wt. % to about 3 wt. % of one or more sweetening agents by weight of the total composition.

In another embodiment, the composition of the present application comprises one or more sweetening agents selected from the group including, but not limited to, stevia, sucrose, glucose, saccharin, levulose, lactose, mannitol, sorbitol fructose, maltose, xylitol, saccharin salts, thaumatin, aspartame, dihydrochalcones, acesulfame, sucralose cyclamate salts, sodium cyclamate, sodium saccharin, and mixtures thereof. In a non-limiting embodiment, the composition contains from about 0.1 wt. % to about 1 wt. %, of one or more sweetening agents by weight of the total composition.

In another embodiment, the composition of the present application comprises lubricants and flavoring oils selected from the group including, but not limited to, avocado oil, coconut oil, palm oil, olive oil, corn oil, sunflower oil, almond oil, canola oil, anise oil, clove oil, sassafras oil, spearmint oil, peppermint oil, oil of wintergreen and mixtures thereof. in a non-limiting embodiment, the composition contains from about 1 wt. % to about 3 wt. % of lubricants and flavoring agents, According to another non-limiting embodiment of the present application, the composition contains from about 0.1 wt. % to about 1 wt. %, of one or, more food grade oils or flavors by weight of the total composition.

Another embodiment of the present application discloses an effervescent co-excipient composition used for (i) enhancing stability of the composition, (ii) developing, free flowing and highly compactible compositions and, (iii) developing effervescent formulations that are shelf stable in standard product packaging. The composion can be used in a standard ready to mix beverages.

Yet another embodiment of the present application discloses a process for co-processing a finely divided admixture comprising individual particle of an effervescent carbonate base or mixture of carbonate bases encapsulated with mannitol or any other water soluble carbohydrate sugar alcohol, wherein the process comprises admixing co-processed base or bases with the mannitol or other water soluble carbohydrate sugar alcohol in solution at 20 to 30% solids and spraying onto the surface of die base or base-mix in an amount sufficient to achieve about an 8:2 weight ratio gain to provide a barrier to prevent premature reaction, drying the resultant encapsulate and dry blending with citric acid co-processed with mannitol.

According to another non-limiting embodiment of the present application, the effervescent co-processed excipient composition can be used in pharmaceutical, food, industrial, biocide, preservative, nutraceutical or agrochemical formulations or compositions. In a non-liming embodiment of the present application, the effervescent co-processed excipient composition can be used in pharmaceutical, food and nutraceutical formulations or compositions.

A different embodiment of the present application discloses an oral solid dosage form comprising: (a) a dry stable effervescent co-processed excipient composition comprising: (i) about 0.1 to about 50 wt. % of one or more carbonate bases selected from the group consisting of sodium bicarbonate (NaHCO3), potassium bicarbonate (KHCO3), sodium carbonate (Na2CO3), potassium carbonate (K2CO3) and combinations thereof: (ii) 0.001 to about 50 wt. % of mannitol: (iii) about 0.001 to about 40 wt. % of one or more organic acids; (b) about to about 3 wt. % of one or More sweeteners selected from the group consisting of stevia, aspartame, sticralose, and saccharin., and (c) about 0.1 to about 1 wt. % of one or more food grade oils or flavors selected from the group consisting of avocado, coconut, palm, olive, corn, sunflower, almond, canola, berry, cherry, passion fruit, orange, blue ice, tropical fruit, raspberry, lemon flavor and mixtures thereof.

In some embodiments, the oral solid dosage form is present in suitable amounts ranging from about 10 wt. % to about 20 or from about 20 wt. % to about 30 wt. %, or from about 30 wt. % to about 40 wt. %, from about 40 wt. % to about 50 wt. %, or from about 50 wt. % to about 60 wt. %, or from about 60 wt. % to about 70 wt. % or from about 70 wt. %) to about 75 wt. % and comprises a dry stable effervescent co-processed excipient composition comprising, (i) about 0.1 to about 50 wt. % of one or more carbonate bases selected from the Group consisting of sodium bicarbonate (NaHCO3), potassium bicarbonate (KHCO3), sodium carbonate (Na2CO3), potassium carbonate (K2CO3) and combinations thereof; (ii) 0.001 to about 50 wt. % of mannitol; and about 0.001 to about 40 wt. % of one or more organic acids.

According to another embodiment of the present application, the oral solid dosage form is in the form of tablets, capsules pellets, mini tablets, granules or a sachet. In some non-limiting embodiments, the present application discloses that the oral solid dosage includes a food, a pharmaceutical, or a nutraceutical ingredient.

In another embodiment, the composition of the present application preferably in oral solid dosage form comprises a flavoring agent which can include, but is not limited to, avocado oil, anise oil, clove oil, sassafras oil, spearmint oil, berry flavor and mixtures thereof. The composition preferably contains such flavoring agent in an amount from about 0.1% to about 1%, by weight of the composition

The following examples are presented for purposes of demonstrating, but not limiting, the preparation and use of the polymers. In the examples, the following abbreviations are used:

| wt % or % (w/w) | Weight percent |
|---|---|
| kPa | Kilopascals |
| kN | Kilonewton |
| kP | Kilopounds |
| D.I. | deionized water |
| DT | disintegration time |
| RH | relative humidity |
| NaHCO₃ | sodium bicarbonate |
| KHCO₃ | potassium bicarbonate |
| Na₂CO₃ | sodium carbonate |
| K₂CO₃ | potassium carbonate |

Further, certain aspects of the present application are illustrated in detail by way of the following examples. The examples are given herein for illustration of the application and are not intended to be limiting thereof.

### EXAMPLES

### Example 1: Manufacturing Procedure For Part A Co-Processed Carbonate Base

Sodium and potassium bicarbonate were combined with potassium carbonate in equal ratios as shown in Table 1 and Table 2 and granulated with mannitol which was added from aqueous solution via top spray granulation in a fluid bed system. Separately, citric acid (Citrocoat-N grade from Jungbunzlauer company) was co-processed in a similar way via top-spray wet granulation with a water dispersed mannitol suspension. After drying and sizing the granulations Table 1 and Table 2 are thy blended together to yield the stabilized, co-processed effervescent excipient in a 60.9: to 39.6 ratio (Table 3), The resultant co-processed excipient was stable at ambient conditions stored in an open dish for 5 days and remains stable packaged in simple PE bags when stored at 25° C. 60% RH and 40° C. 75% RH.

Composition of co-processed carbonate component

| Ingredients | W/W (%) |
|---|---|
| Na₂CO₃ | 30.0 |
| NaHCO₃ | 10.0 |
| KHCO₃ | 30.0 |
| K₂CO₃ | 10.0 |
| Mannitol | 20.0 |
| Total | 100.0 |

TABLE 2
Composition of co-processed citric acid component

| Ingredients | W/W (%) |
|---|---|
| Citric acid | 80.0 |
| Mannitol | 20.0 |
| Total | 100.0 |

Composition of the stable effervescent co-processed excipient granulation was based on the stoichiometric ratios of bicarbonate, carbonate and citric as per the reactions below:

One H₃C₆H₅O₇ (citric acid)+3 alkali HCO₃=Alkali carbonate salt+3 CO₂+H₂O

Two H₃C₆H₅O₇ (citric acid)+3 alkali CO₃=two Alkali carbonate salt+3 CO₂+3 H₂O

Three moles of alkali bicarbonate require one mole of citric acid and two moles of alkali carbonate requires two moles of citric acid. As a result, the final composition of stable effervescent co-processed excipient granulation contains 50-75% of co-processed carbonate base and 25-40% of co-processed citric base. Table 3 shows one example of stable effervescent co-processed excipient effervescent granulation.

TABLE 3
Composition of stable effervescent
co-processed excipient

| Ingredients | W/W (%) |
|---|---|
| Co-processed Carbonate | 60.4 |
| component | |
| Co-processed Citric acid | 39.6 |
| component | |
| Total | 100.0 |

TABLE 4
Stable effervescent
co-processed excipient—Formulation

| Ingredients | W/W (%) |
|---|---|
| Na₂CO₃ | 18.12 |
| NaHCO₃ | 6.04 |
| KHCO₃ | 18.12 |
| K₂CO₃ | 0.04 |
| Citric Acid | 31.68 |
| Mannitol | 20.0 |
| Total | 100.0 |

### Example 2: Water Activity After 5 Days Of Stable Effervescent Co-Processed Excipient And

### Uncoated Effervescent Blend

Several stable effervescent co-processed exciptent samples and uncoated effervescent blends were stored at different conditions: at ambient open dish and in a simple polyethylene bag at 25° C. 60% RH and 40° C., 75% RH for 5 days.

Water activity of each uncoated effervescent blend and stable effervescent co-processed excipient samples were measured using an AquaLab instrument which measures the energy status of the water in the sample (FIG. 9).

Table 5 shows proprietary stable effervescent co-processed excipient that has unchanged water activity as uncoated ingredients after 5 days at various challenging storage conditions. That indicates that the stable effervescent excipient can prevent any self-propagation between the acid and carbonate until the time of intended use without being influenced by free surface water.

TABLE 5
Water Activity after 5 days of stable effervescent co-processed excipient
and Uncoated Effervescent Blend

| | | Stable effervescent co- |
|---|---|---|
| | Control* | processed excipient |
| Sample Type | Water Activity | Water Activity |
| Initial | 0.552 | 0.501 |
| Room Temp for 5 Days (open dish) | 0.617 | 0.529 |
| 25° C./60% RH for 5 days | 0.696 | 0.472 |
| 40° C./75% RH for 5 days | 0.705 | 0.492 |

*Control = same ingredients without proprietaty processing

### Example 3: Electrolyte Replacement Tablet Formulation

The stable effervescent co-processed excipient was blended with an electrolyte replacement blend and other ingredients (Table 6) to make an effervescent electrolyte replacement tablet formulation.

TABLE 6
Electrolyte replacement effervescent tablet formulation

| | Tablet weight | Tablet Weight |
|---|---|---|
| Ingredients | (w/w %) | (mg) |
| Electrolyte replacement Blend | 30.0 | 750.0 |
| Stable effervescent co-processed excipient | 66.0 | 1650.0 |
| Stevia sweetener | 3.0 | 75.0 |
| Avocado oil | 1.0 | 25.0 |
| Total | 100.0 | 2500.0 |

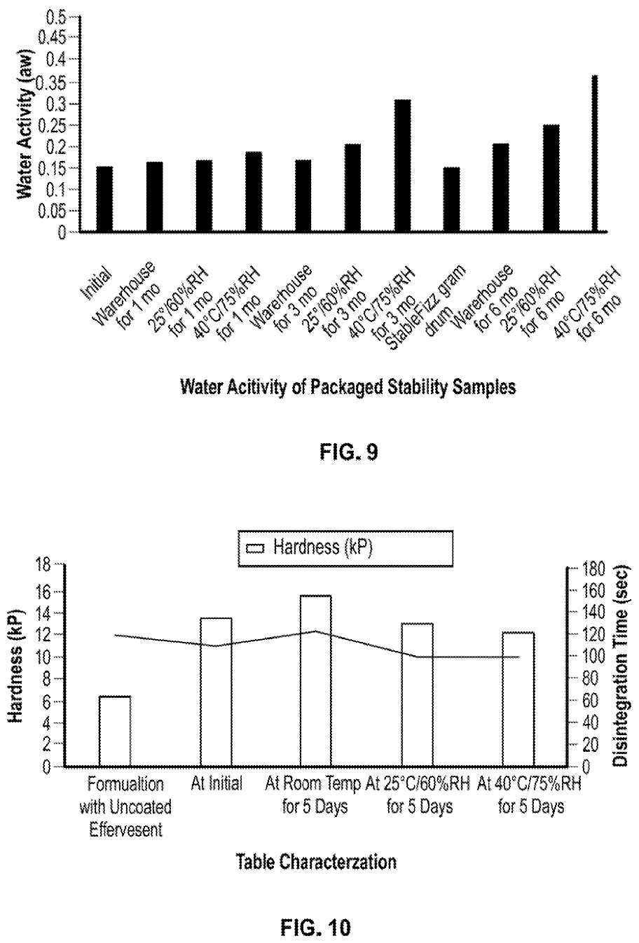

Tablet characterization at compression force of 50 kN and tablets with effervescent granulation maintain hardness and compression strength (Table 6a) and use level of tablets (Table 6b and Table 6c) given in FIG. 10.

TABLE 6a
Electrolyte replacement effervescent tablet

| | Hardness | Disintegration |
|---|---|---|
| Tablet ID | (kp) | Time (Sec) |
| Formulation with uncoated effervescent blend | 6.5 | 120 |
| At initial | 13.5 | 110 |
| At room temp for 5 days | 15.5 | 123 |
| At 25° C./60% RH for 5 days | 13.04 | 100 |
| At 40° C./75% RH for 5 days | 12.26 | 100 |

TABLE 6b
Use levels of effervescent drink volume

| Effervescent | |
|---|---|
| Drink Volume | Use Level |
| 8 OZ-10 OZ | 60-70% |
| 5 OZ-6 OZ | 40-50% |
| 3 OZ-4 OZ | 20-25% |

TABLE 6c
Use Levels of effervescent table

| Effervescent | | |
|---|---|---|
| Tablet | Use | Water |
| Weight | Level | Volume |
| 3-4 GM | 60-70% | 6 OZ |
| 1-2 GM | 40-50% | 6 OZ |

Tablet hardness for the electrolyte replacement effervescent tablets were tested using Natoli automated tablet hardness tester. The disintegration time of each tablet was also measured in deionized water at 37° C. (Table 7).

TABLE 7
Tablet characterization at compression force of 50 kN

| | Hardness | DT |
|---|---|---|
| Tablet ID | (kP) | (Sec) |
| At initial | 13.5 | 110.0 |
| At room temp for 5 days | 15.5 | 123.0 |
| At 25/60 for 5 days | 13.04 | 100.0 |
| At 40/75 for 5 days | 12.26 | 100.0 |

### Example 4: Tart Cherry Extract Tablet Formulation

Similar to the electrolyte replacement effervescent tablet formulations, the stable effervescent co-processed was blended with tart cherry extract, and other ingredients to make a tart cherry extract effervescent tablet formulation (Table 8).

TABLE 8
Tablet cherry extract tablet formulation

| | Tablet | Tablet |
|---|---|---|
| | weight | Weight |
| Ingredients | (w/w %) | (mg) |
| Tart cherry extract | 20.0 | 500.0 |
| Stable effervescent excipient | 75.0 | 1875.0 |
| Stevia sweetener | 3.0 | 75.0 |
| Mixed berry flavor | 1.0 | 25.0 |
| Avocado oil | 1.0 | 25.0 |
| Total | 100.0 | 2500.0 |

Tablet hardness of each tart cherry extract. effervescent tablet formulation was tested using Natoli automated tablet hardness tester. The disintegration time of each tablet was also measured in deionized water at 37° C. (Table 9).

TABLE 9
Tablet characterization at compression force of 50 kN

| Tablet ID | Hardness (kP) | DT (Sec) |
|---|---|---|
| At initial | 17.5 | 94.0 |
| At room temp for 5 days | 17.0 | 120.0 |
| At 25/60 for 5 days | 18.0 | 122.0 |
| At 40/75 for 5 days | 17.0 | 110.0 |

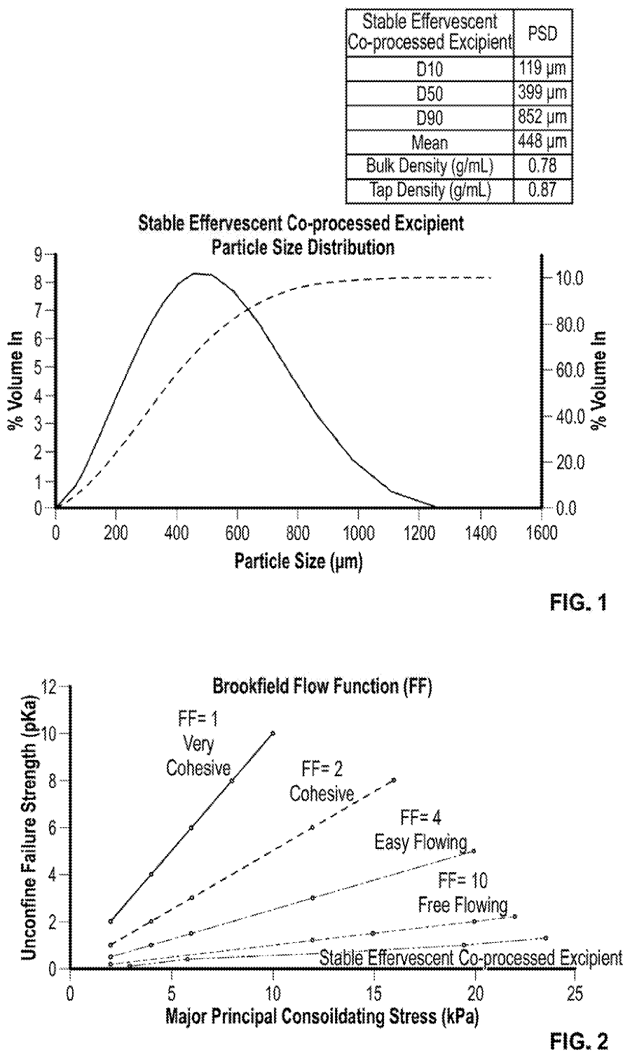

Particle size distribution of the stable effervescent co processed excipient was measured using Malvern Mastersizer 3000 at (470 nm) laser power of 71.35% and beam length of 10.0 nm. FIG. 1 shows stable effervescent co-processed excipient has a very uniform particle size distribution. Stable effervescent co-processed excipient powder flowability was measured using Brookfield Engineering Lab Instrument maximum stress 13.252 kPa with axial speed 1.0 mm/sec and rotational speed 1.0 rev/hr.

Stable effervescent co-processed excipient has excellent powder flow for low variability during direct compression tableting process (FIG. 2). Vapor absorption of the stable effervescent co-processed excipient was measured at different humidity condition using TGA Q5000 Instrument with the method log: Equilibrate at 60.00° C.: Humidity 0.00%; Abort next iso if Weight (%)<0.0100 for 15.00 min; Isothermal for 1440.00 min; Mark data; Equilibrate at 25.00° C.; Humidity 10.00%; Abort next iso if Weight (%)<0.0100 for 15.00 min; Isothermal for 1440.00 min; Mark data; Abort next iso if Weight (%)<0.0100 for 15.00 min; Step humidity 10.00% every 1440.00 min to 90.00%.

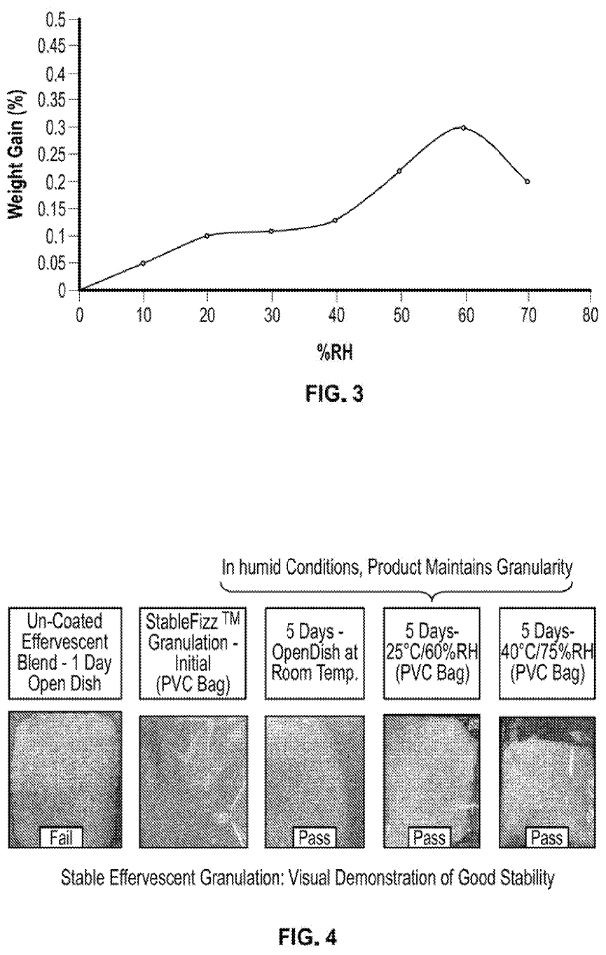

Stable effervescent co-processed excipient has low moisture uptake in humid environments (less than 0.3% at 60% RH) (FIG.3).

Stable effervescent co-processed excipient shows visual demonstration of good stability (FIG. 4).

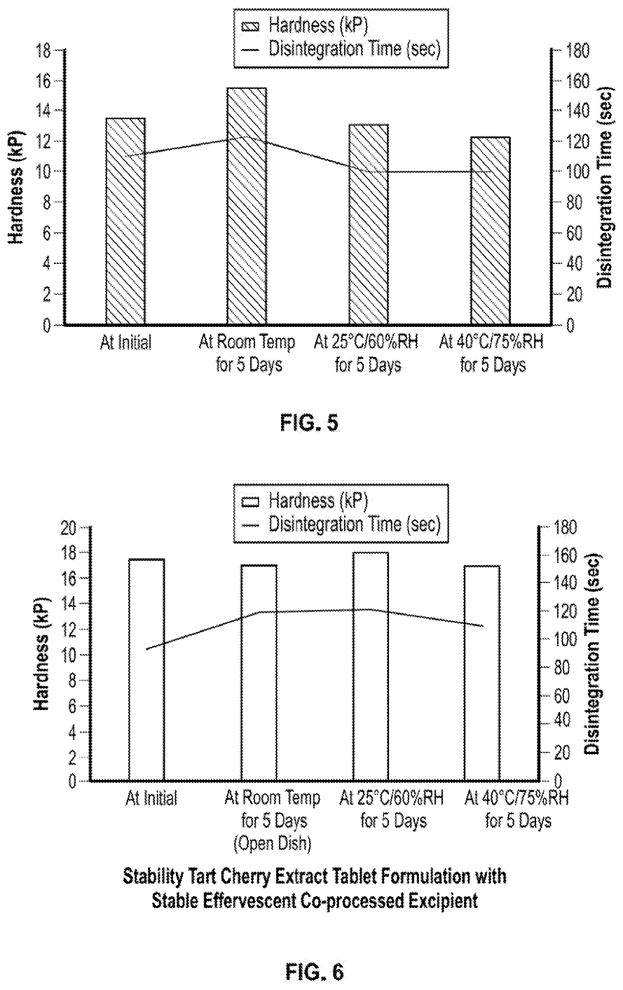

The electrolyte replacement blend and tart Cherry extract effervescent tablet formulations with stable effervescent co-processed effervescent excipient show similar tablet hardness and disintegration time at different stability conditions. The disintegration time of each tablet was measured in deionized water at 37° C. All tablet formulations have tablet hardness around 14 kp and disintegration time of 120-150 seconds, indicating electrolyte tablet formulations with different stable effervescent co-processed excipient samples which were exposed at different stress conditions have good tablet formulation stability (FIG. 5 and FIG. 6).

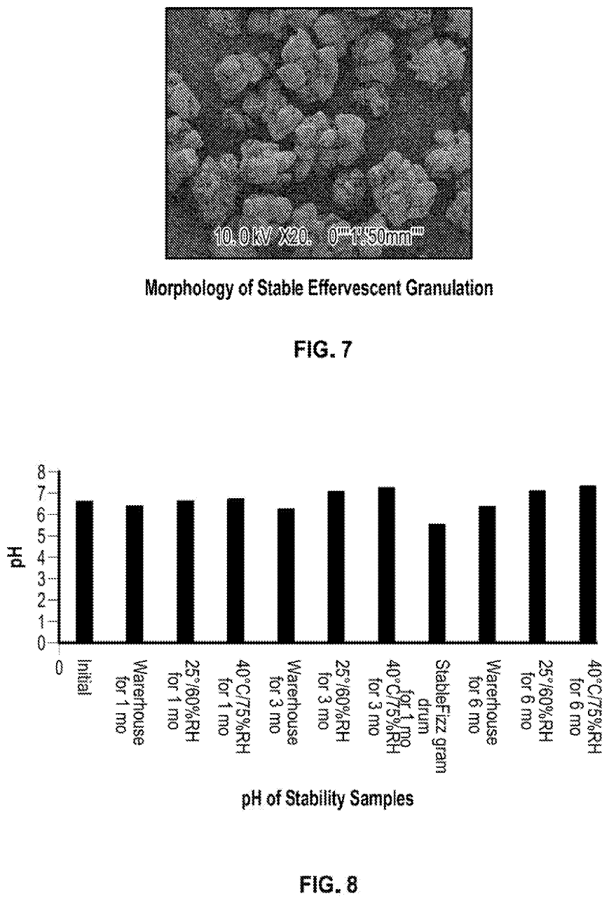

The sample was mounted on a sample stub, coated with a thin layer of Au/Pd to make the sample surface conductive and then examined in. SEA (Secondary Electron Imaging) mode. SEI records the topographical features of the sample surface. Representative photomicrographs were digitally captured at 2048×1594 pixel resolution. The samples were examined at multiple magnifications and areas. The image was presented in the FIG. 7.

Using USP 791 method, pH of stability samples show the minimal change in pH, indicating the stable effervescent granulation maintained its chemical properties (FIG. 8).

Using USP 922 water activity method, water activity of packaged stability samples are examined, at the 40° C./75%RH samples as shown in FIG. 9 had the highest water activity, indicating the stable effervescent granulation was most stable at room temperature (warehouse) or 25° C./60% RH conditions.

While the compositions and methods of the disclosed and/or claimed inventive concepts have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concepts. All such similar substitutes and modifications apparent to those skilled in the art are deemed to be within the spirit, scope and concept of the disclosed and: or claimed inventive concepts.

What is claimed is:

1. A dry stable effervescent co-processed excipient composition comprising:
i. about 0.1 to about 50 wt. % of one or more carbonate bases selected from the group consisting of alkali carbonate, alkali bicarbonate, alkaline earth carbonate, alkaline earth bicarbonate and mixtures thereof;
ii. about 0.1 to about 50 wt. % of one or more water soluble carbohydrate sugar alcohols selected from the group consisting of mannitol, lactitol, erythritol and mixtures thereof; and
iii. about 0.001 to about 40 wt. % of one or more organic acids.

2. The effervescent co-processed excipient composition according to claim 1, wherein the carbonate base is selected from the group consisting of sodium bicarbonate (NaHCO₃), potassium bicarbonate (KHCO₃), sodium carbonate (Na₂CO₃), potassium carbonate (K₂CO₃) and combinations thereof.

3. The effervescent co-processed excipient composition according to claim 1, wherein said organic acid is selected from the group consisting of citric acid, malic acid, tartaric acid and mixtures thereof.

4. The effervescent co-processed excipient composition according to claim 1 used for (i) enhancing stability of the composition, (ii) developing a free flowing and highly compactible composition and, (iii) developing an effervescent formulation that is shelf stable in a standard product packaging and standard ready to mix beverages.

5. A process for co-processing a finely divided admixture comprising individual particles of an effervescent carbonate base or mixture of carbonate bases encapsulated with mannitol, wherein the process comprises admixing a co-processed base or bases with mannitol in solution at 20-30% solids and spraying onto the surface of the base or base-mix in about 8:2 weight ratio gain to provide a barrier to prevent premature reaction, drying the resultant encapsulate and dry blending with citric acid co-processed with mannitol.

6. The effervescent co-processed excipient composition of claim 1 used for preparing a tablet, a capsule, a pellet, a mini tablet or a sachet.

7. The effervescent co-processed excipient composition of claim 1 used in pharmaceutical, food, industrial, biocide, preservative, or agrochemical formulations.

8. An oral solid dosage form comprising:
a) about 10 to about 75 wt. % of a dry stable effervescent co-processed excipient composition comprising (i) about 0.1 to about 50 wt. % of one or more carbonate bases selected from the group consisting of sodium bicarbonate (NaHCO₃), potassium bicarbonate (KHCO₃), sodium carbonate (Na₂CO₃), potassium carbonate (K₂CO₃) and combinations thereof, (ii) 0.001 to about 50 wt. % of mannitol; (iii) about 0.001 to about 40 wt. % of one or more organic acids;
b) about 1 to about 3 wt. % of one or more sweeteners selected from the group consisting of stevia, aspartame, sucralose, and saccharin; and
c) about 0.1 to about 1 wt. % of one or more food grade oils or flavors selected from the group consisting of avocado, coconut, palm, olive, corn, sunflower, almond, canola, berry, cherry, passion fruit, orange, blue ice, tropical fruit, raspberry, lemon flavor and mixtures thereof.

9. The oral solid dosage form according to claim 8, wherein the sweetener is stevia.

10. The oral solid dosage form according to claim 8, wherein the food grade oil or flavor is avocado or berry flavor.

11. The oral solid dosage form according to claim 8, wherein the oral solid dosage form is in the form of a tablet, a capsule, a pellet, a mini tablet, or a granule or a powder.

12. The oral solid dosage form according to claim 8, wherein the oral solid dosage includes a food, a pharmaceutical, or a nutraceutical ingredient.
